(19) United States
(12) Patent Application Publication
(10) Pub. No.: US 2005/0089516 A1
(43) Pub. Date: Apr. 28, 2005

(54) Skin vitalizing composition for external use anti-aging preparation

(75) Inventors: Satoshi Amano, Yokohama-shi (JP); Yuki Ogura, Yokohama-shi (JP); Yukiko Matsunaga, Yokohama-shi (JP); Takanari Tsuda, Yokohama-shi (JP); Yukari Aoyama, Yokohama-shi (JP); Nobuyoshi Koga, Yokohama-shi (JP)

Correspondence Address: FOLEY AND LARDNER;SUITE 500, 3000 K STREET NW, WASHINGTON, DC, 20007, US

(73) Assignee: SHISEIDO COMPANY, LTD.

(21) Appl. No.: 10/931,252

(22) Filed: Sep. 1, 2004

Related U.S. Application Data
(63) Continuation of application No. 10/314,165, filed on Dec. 9, 2002, now abandoned.

(30) Foreign Application Priority Data
Jun. 18, 2002 (JP) 2002-177601
Nov. 6, 2002 (JP) 2002-323030

(51) Int. Cl. A61K 38/48

(52) U.S. Cl. 424/94.64

(57) ABSTRACT

The invention provides an epidermal basement membrane structure formation accelerating preparation and a skin external preparation comprising a serine protease inhibitor, and optionally an accelerator of production of extracellular matrix protein components of the epidermal basement membrane. It also provides, as a means for producing artificial skin having an adequately formed basement membrane, an artificial skin-forming medium which comprises a serine protease inhibitor, and optionally an accelerator of production of extracellular matrix protein components of the epidermal basement membrane and a matrix metalloprotease inhibitor, as well as a method for producing the same.

### FIELD OF THE INVENTION

The present invention relates to a skin basement membrane care product, a skin basement membrane structure formation and repair-accelerating preparation, as well as a skin external preparation. The invention also relates to an artificial skin structure formation accelerating preparation and an artificial skin production method.

### PRIOR ART

In the fields of cosmetics and dermatology, a number of various means have been proposed and attempted for alleviation or treatment of skin damage caused by effects of the external environment, including sunlight rays, as well as aging. The major changes in skin that occur with aging are, for example, wrinkle formation, hardening and elasticity loss.

Attention has become focused on the causes of such changes, which include hypofunction of collagen and elastic fibers composed of collagen, elastin and glucosaminoglycans in the dermis. To date, the use of hydroxycarboxylic acids (e.g., Japanese Patent Publication No. 253339) and the use of lysophospholipids (e.g., Japanese Unexamined Patent Publication (KOKAI) No. 8-67621) have been investigated as means for preventing and repairing such skin alterations.

The former publication suggests that cornification or wrinkle formation can be inhibited by preventing loss of collagen fibers. The latter publication, on the other hand, suggests that lysophospholipids have a moisturizing effect in the dermis through accelerating the production of glucosaminoglycans (specifically, hyaluronic acid) in human fibroblasts.

In general terms, skin consists of a horny layer, epidermis, basement membrane and dermis. Type IV and Type VII collagens are components of the epidermal basement membrane responsible for binding the epidermis to the dermis. Type IV collagen is the major component of the lamina densa structure forming the skeleton of the epidermal basement membrane. Type VII collagen is the major component of the anchoring fibrils that bind the basement membrane to the dermis.

It has been observed that the level of expression of Type IV collagen in the epidermal basement membrane decreases with aging (Vazquez F. et al., Maturitas 1996, 25:209-215), while some reports indicate that Type VII collagen production on the protein level and mRNA level also decreases in skin fibroblasts taken from elderly persons compared to skin fibroblasts from younger individuals (Chen et al., J. Invest. Dermatol., 102:205-209, 1994). Also, reduction of the anchoring fibrils composed of Type VII collagen have been reported to occur with physiological aging and photoaging of normal skin (Tsuji, T., Nippi Kaishi 105:963-975, 1995; Tidman et al., J. Invest. Dermatol., 83:448-453, 1984). Thus, acceleration of the formations of Type IV collagen that forms the basement membrane skeleton, anchoring fibrils that bind the epidermis to dermis, as well as Type VII collagen which is the major component of the anchoring fibrils, are deemed as important factors for maintaining healthy and youthful skin.

The most powerful factor in the external environment which affects skin aging is ultraviolet rays included in sunlight, which have been clearly established as an aging-accelerating factor and are known to induce skin changes, so-called photoaging, characterized by deep wrinkles (Scharffetter-Kochanek, Advance in Pharmacology, 1997, 58, 639-655). Ultraviolet rays have various effects on the skin, which include damage to genetic DNA, induced production of active oxygen and, as recently demonstrated, induced production of matrix metalloproteases (Fischer et al., Nature, 1996, 379, 335-339).

Due to the multipotent nature of ultraviolet rays, the mechanisms by which ultraviolet ray-induced photoaging occurs have not yet been adequately elucidated. Experiments involving continuous irradiation of hairless mice with ultraviolet rays at an energy dose which does not cause erythema have demonstrated that deep wrinkles are formed in the mouse dorsal skin corresponding to human photoaged skin, and such mouse models have been used to evaluate substances that can affect wrinkling (Moloney et al., Photochem. Photobiol., 1992, 56, 495-504). However, the mechanisms of wrinkle formation have not yet been adequately elucidated.

On the other hand, Koivulkangas et al. have reported that the activity of the basement membrane catabolic enzyme gelatinase increases in ultraviolet-irradiated skin (Acta Derm. Venereol. 1994, 74, 279-282). Also, Marschall et al. have reported an increase in expression of urokinase (plasminogen activator (uPA)) and its receptor, uPA receptor, in epidermal cells subjected to ultraviolet irradiation (J. Invest. Dermatol. 1999, 113, 69-76), suggesting that supplement of blood plasma-derived plasminogen activates plasminogen surrounding keratinocytes and increases plasmin activity at sites of ultraviolet ray exposure. It has also been reported that the basement membrane exhibits structural changes in sunlight-exposed sites of skin, and particularly extensive multilayering is frequently observed (Lavker, J. Invest. Dermat., 1979, 73, 59-66). This suggests that ultraviolet rays included in sunlight affect the basement membrane structure by increasing production of catabolic enzymes in the skin basement membrane.

Damage to the epidermal basement membrane not only accelerates skin aging but also impedes the daily turnover of epidermis, eliciting further skin damage. Rapid repair of the structure of damaged skin basement membrane is therefore important not only for preventing skin aging, but also as a part of routine skin care.

Specific means for accelerating repair of the basement membrane have been investigated using artificial skin models. As exemplified method for producing an artificial skin model includes culturing normal human epidermal keratinocytes on human fibroblast-containing contracted Type I collagen gel, which has a dermis-like structure, to form an epidermal layer. In this method, a defective basement membrane will be formed between the dermal-imitating collagen gel and the epidermal-imitating layer, and it can therefore be used to evaluate substances that accelerate repair or regeneration of damaged skin basement membrane structure. By using such an artificial skin, it was discovered that acceleration of the regeneration of the skin basement membrane structure can be accomplished by applying a matrix metalloprotease inhibitor, or both the matrix metalloprotease inhibitor and a matrix protein production promoter (Japanese Unexamined Patent Publication (KOKAI) No. 2001-269398). However, it has been a problem that the structure of the regenerated lamina densa is underdeveloped in comparison to normal skin, and the depositing of Type VII collagen is also inadequate.

Artificial skin is important as a substitute for biological skin which has been damaged by any cause, or as an experimental material for testing the effects of skin medicines and cosmetics, or for drug testing, and for all such uses, it is desirable for the artificial skin to have a structure resembling as closely as possible the natural skin structure.

It is an object of the present invention to provide a new means and skin external preparation for accelerating repair and regeneration of the skin basement membrane structure, as well as a new method for achieving adequate formation of basement membrane in artificial skin structures.

### DISCLOSURE OF INVENTION

The present inventors have conducted diligent research directed toward solving the problems associated with accelerating repair and regeneration of the skin basement membrane structure and with production of artificial skin having a satisfactory basement membrane structure, and as a result we have completed the present invention upon finding that the basement membrane formation-accelerating effect of matrix metalloprotease inhibitors is notably enhanced by substances that inhibit serine proteases and by substances that increase production of Type IV and Type VII collagen or laminin 5, which are major components of the epidermal basement membrane.

The invention therefore provides a skin vitalizing composition for external use anti-aging preparation, in particular, an epidermal basement membrane structure formation accelerating preparation and a skin external preparation comprising one or more serine protease inhibitors as the active ingredients.

The invention further provides an epidermal basement membrane structure formation accelerating preparation and a skin external preparation, each formulated as a mixture of one or more serine protease inhibitors, and one or more substances that increase production of the extracellular matrix components, for example, the major epidermal basement membrane component Type IV or Type VII collagen or laminin 5.

The invention still further provides an epidermal basement membrane structure formation accelerating preparation and, a skin external preparation, each formulated as a mixture of one or more serine protease inhibitors, one or more substances that increase production of the extracellular matrix components, for example, the major epidermal basement membrane component Type IV or Type VII collagen or laminin 5, and also, one or more matrix metalloprotease inhibitors.

The invention yet further provides as a means for producing artificial skin having an adequately formed basement membrane, an artificial skin-forming medium which comprises one or more serine protease inhibitors, one or more substances that increase production of the extracellular matrix components, for example, the major epidermal basement membrane component Type IV or Type VII collagen or laminin 5, and/or one or more matrix metalloprotease inhibitors, as the active ingredients, either alone or in admixture, as well as a method for producing the medium.

The present inventors have found that by combining an extract derived from a plant belonging to Fagaceae Fagus as the substance exhibiting Type IV and Type VII collagen production promoting activity, 1-acyl lysophospholipid as the laminin 5 production promoter, and an extract derived from a plant belonging to Labiatae Mentha as the serine protease inhibitor, it is possible to obtain a skin external preparation which exhibits a high basement membrane repair-accelerating effect, an excellent rough skin-ameliorating effect and an excellent skin elasticity-maintaining effect, and inhibits wrinkle formation.

More specifically, according to a preferred mode of the present invention, there is provided a skin external preparation characterized by comprising an extract derived from a plant belonging to Fagaceae Fagus, 1-acyl lysophospholipid represented by the following general formula (1) or (2), and an extract derived from a plant belonging to Labiatae Mentha: 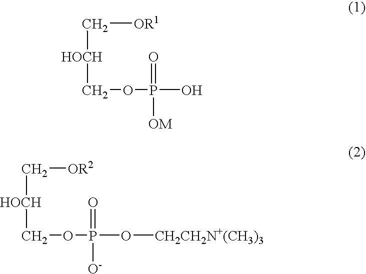
where R¹ represents a saturated fatty acid residue having 11-24 carbon atoms or a fatty acid residue having 18, 20, 22 or 24 carbon atoms and 1-4 unsaturated double bonds, R² represents a saturated fatty acid residue having 13-24 carbon atoms or a fatty acid residue having 18, 20, 22 or 24 carbon atoms and 1-4 unsaturated double bonds, and M represents H or an alkali metal atom.

### BRIEF DESCRIPTION OF DRAWINGS

FIG. 1 is a cross-sectional view of artificial skin showing a comparison of culturing to form artificial skin without addition (1) and with addition (2) of the matrix metalloprotease compound A to the medium.

FIG. 2 is a cross-sectional view of artificial skin showing a comparison of culturing to form artificial skin with addition of 10 μM of compound A and 10 μg/ml of aprotinin (3), 10 μM of compound A and 1 ng/ml of interleukin-1β (4), and 10 μM of compound A, 1 ng/ml of interleukin-1β and 10 μg/ml of aprotinin (5) to the medium.

FIG. 3 is a cross-sectional view of artificial skin showing a comparison of culturing to form artificial skin with addition of 10 μM of compound A and 1 ng/ml of TGF-α (6), and 10 μM of compound A, 1 ng/ml of TGF-α and 10 μg/ml of aprotinin (7) to the medium.

FIG. 4 is a cross-sectional view of artificial skin showing a comparison of culturing to form artificial skin with addition of 10 μM of compound A and 100 ng/ml of PDGF (8) and 10 μM of compound A, 100 ng/ml of PDGF and 10 μg/ml of aprotinin (9) to the medium.

FIG. 5 is a transmission electron microscope photograph of artificial skin showing a comparison of culturing to form artificial skin with addition of the matrix metalloprotease inhibitor compound A alone (1) and with further addition of the serine protease inhibitor aprotinin (2) to the medium.

FIG. 6 is a hematoxylin and eosin stained cross-sectional view of artificial skin showing a comparison of culturing to form artificial skin with (1) no addition (control), and addition of (2) soybean lysolecithin, (3) peppermint extract, (4) beech bud extract or (5) soybean lysolecithin+peppermint extract+beech bud extract to the medium.

FIG. 7 is an immunostained cross-sectional view of artificial skin showing a comparison of culturing to form artificial skin with (1) no addition (control), and addition of (2) soybean lysolecithin, (3) peppermint extract, (4) beech bud extract or (5) soybean lysolecithin+peppermint extract+beech bud extract to the medium.

FIG. 8 is a cross-sectional tissue image of artificial skin transplanted into nude mice and collected at day 7 following the transplantation, where the artificial skin had been cultured with no additives or in the presence of the matrix metalloprotease inhibitor compound A (10 μM) and the serine protease inhibitor aprotinin (10 μg/ml). It shows tissue images of the artificial skin before transplantation (laminin 5 staining) and the adhered artificial skin (hematoxylin and eosin staining, laminin staining, as well as 2-microglobulin staining).

FIG. 9 is a photograph of the outer appearance of artificial skin transplanted into nude mice and collected at days 7 and 18 following the transplantation, where the artificial skin had been cultured with no additive or in the presence of the matrix metalloprotease inhibitor compound A (10 μM) and the serine protease inhibitor aprotinin (10 μg/ml).

### BEST MODE FOR CARRYING OUT THE INVENTION

Serine Protease Inhibitors

There are no particular restrictions on the serine protease-inhibiting substances to be used for the invention so long as they are substances which exhibit such inhibitory activity. Examples of serine proteases include plasmin, urokinase and the like. Thus, the serine protease-inhibiting substances may be selected from among substances that inhibit plasmin, urokinase and the like.

As specific examples of serine protease-inhibiting substances, there may be mentioned aprotinin, tranexamic acid and ε-aminocaproic acid, or the like.

Specific examples of serine protease inhibitors that may be used according to the invention include various plant extracts that exhibit serine protease-inhibiting activity, and their purified products. As such plants there may be mentioned Calophyllum brasiliense Cambess., Myrcia sphaerocarpa DC, Hyptis crenata Pohl ex Benth., C. rotundus L, E. sylvestris var. ellipticus and E. decipiens Helmsl.; plants belonging to the Rosaceae family, and especially plants belonging to the genera Rosa, Rubus, Filipendula and Crataegus of the Rosaceae family; plants belonging to the family Paeoniaceae, genus Paeonia; plants belonging to the family Moraceae, genus Humulus, plants belonging to the family Compositae, genus Arnica and the family Compositae, genus Anthemis, plants belonging to the family Pyrolaceae, plants belonging to the family Hypericaceae such as Hypericium erectum T. and Hypericium perforatum, plants belonging to the family Labiatae, genus Mentha such as Mentha piperata L. and Mentha viridis L.; plants belonging to Compositae Carthamus tinctorius L., Geraniaceae Geranium thunbergii Siebold et Zuccarini and Vitaceae Vitis; plants belonging to the family Betulaceae such as Betula alba L., Betula lutea L. and Betula pendula Roth.; plants belonging to the family Rosaceae, genus Prunus, which are apricots (P. armeniaca L., P ansu Komar, P. mandshurica Koehne, P. sibirica L.); and plants which grow in dry prairies or grasslands, such as zapote, que shar, sano-sano, cora de cavallo, and the like.

Such plant extracts may be obtained from the roots, leaves, stems, flowers, etc. of herbal plants or from the roots, buds, bark, fruit, leaves, flowers, etc. of woody plants.

The extracts derived from these plants may be obtained by drying the plant material, if necessary, and further slicing or pulverizing it, if necessary, and then using cold water, warm water or boiling/hot water to prepare aqueous extracts, while organic solvents such as methanol, ethanol, 1,3-butanediol and ether may also be used therewith at ambient temperature or with heating.

Extracellular Matrix Protein Production Accelerators

The extracellular matrix protein production accelerators to be used for the invention are substances which accelerate the production of such proteins.

As matrix proteins according to the present invention, there may be mentioned the basement membrane components laminin, Type VII collagen, perlecan, nidogen and the like, and especially laminin 5, Type IV collagen and Type VII collagen, which are distinctive components of the skin basement membrane.

As specific examples of laminin 5 production accelerators, there may be mentioned transforming growth factor-α, transforming growth factor-β1, transforming growth factor-β2, transforming growth factor-β3, epidermal growth factor, and the like.

Various plant extracts and their purified forms may also be used. As such plants, there may be mentioned glycyrrhiza, blackberry lily, Alstonia scholaris, Tinospora crispa, fenugreek, Papaveraceae Bocconia palo amarillo, Leguminosae Psophocarpus tetragonolobus, Leguminosae Cassia retama, Gentianaceae Erythraea chilensis (canchalagua), soybean, pueraria root, cammock, melilot, sprouts, red bean, and the like. These plant extracts may be obtained from the roots, leaves, stems, flowers, etc. of herbal plants or from the roots, buds, bark, fruit, leaves, flowers, etc. of woody plants.

The extracts from these plants may be obtained by drying the plant material, if necessary, and further slicing or pulverizing it, if necessary, and then using cold water, warm water or boiling/hot water to prepare aqueous extracts, while organic solvents such as methanol, ethanol, 1,3-butanediol and ether may also be used therewith at ordinary temperature or with heating. Purified extracts from such plants include soybean lysolecithin, soybean saponin fragments, soybean lecithin fragments, and the like.

As specific examples of Type IV collagen production accelerators there may be mentioned tumor necrosis factor-α, transforming growth factor-β1, transforming growth factor-β2, transforming growth factor-β3, epidermal growth factor, interleukin-1α (IL-1α), interleukin-1β (IL-1β), platelet-derived growth factor, and the like.

Various animal-derived materials may also be used. For example, there may be mentioned bromois milk prepared from milk casein, EM protein L prepared by hydrolysis of chicken egg shell membrane, and the like.

Various plant-derived extracts and their purified products may also be used. As such plants there may be mentioned plants belonging to the family Apocynaceae such as pule (Alstonia scholaris), trees belonging to the family Fagaceae such as Fagus sylvatica, plants of the family Leguminosae such as Pueraria lobata Ohwi, plants of the family Araliaceae such as Hedera helix, and plants such as orchids and the like. Such plant extracts may be obtained from the roots, leaves, stems, flowers, etc. of herbal plants or from the roots, buds, bark, fruit, leaves, flowers, etc. of woody plants.

The extracts from these plants may be obtained by drying the plant material, if necessary, and further slicing or pulverizing it, if necessary, and then using cold water, warm water or boiling/hot water to prepare aqueous extracts, while organic solvents such as methanol, ethanol, 1,3-butanediol and ether may also be used therewith at ambient temperature or with heating.

As specific examples of Type VII collagen production accelerators, there may be mentioned tumor necrosis factor-α, transforming growth factor-β1, transforming growth factor-β2, transforming growth factor-β3, epidermal growth factor, interleukin-1α (IL-1α), interleukin-1β, platelet-derived growth factor, and the like.

Various animal-derived substances may also be used. For example, there may be mentioned bromois milk prepared from milk casein, EM protein L prepared by hydrolysis of chicken egg shell membrane, and the like.

Various plant-derived extracts and their purified products may also be used. As such plants there may be mentioned plants belonging to the family Apocynaceae such as pule (Alstonia scholaris), trees belonging to the family Fagaceae such as Fagus sylvatica, plants of the family Leguminosae such as Pueraria lobata Ohwi, plants of the family Araliaceae such as Hedera helix, and plants such as orchids and the like. Such plant extracts may be obtained from the roots, leaves, stems, flowers, etc. of herbal plants or from the roots, buds, bark, fruit, leaves, flowers, etc. of woody plants.

The extracts from these plants may be obtained by drying the plant material, if necessary, and further slicing or pulverizing it, if necessary, and then using cold water, warm water or boiling/hot water to prepare aqueous extracts, while organic solvents such as methanol, ethanol, 1,3-butanediol and ether may also be used therewith at ambient temperature or with heating.

Matrix Metalloprotease Inhibitors

There are no particular restrictions on the matrix metalloprotease inhibitors that may be used for the present invention, so long as they are substances which exhibit such inhibitory activity. Examples of matrix metalloproteases include gelatinase, collagenase, stromelysin, matrilysin, and the like. Thus, the matrix metalloprotease inhibitors may be selected from among substances that inhibit gelatinase, collagenase, stromelysin, matrilysin, and the like.

As specific examples of matrix metalloprotease inhibitors, there may be mentioned N-hydroxy-2-[[(4-methoxyphenyl)sulfonyl]-3-picolyl]amino)]-3-methyl butaneamide hydrochloride (hereinafter referred to as “Compound A”) (J. Med. Chem. 1997, 40, p.2525-2532), MMP-inhibitor (p-NH₂-Bz-Gly-Pro-D-Leu-Ala-NHOH) (FN-437) (BBRC, 1994, 199, 1442-1446), etc.

Specific examples of matrix metalloprotease inhibitors according to the present invention include various plant extracts and their purified products. As such plants there may be mentioned Thymus serpyllum L., Valeriana fauriei Briquet and other plants of the family Valerianaceae, Diospyros kaki Thunberg (family Ebenaceae), Astragalus sinicus Linne (family Leguminosae), Crataegus cuneata Siebold et Zuccarini (family Rosaceae), Paeonia suffruticosa Andrews (Poeonia montan Sims) (family Paconiaceae), Thea sinensis Linne var. assamica Pierre, (family Theaceae), Eucalyptus globulus Labillardiere and other plants of the family Myrtaceae, Potentilla tormentilla Schrank (family Rosaceae), Tilia cordata Mill., Tilia platyphyllus Scop., Tilia europaea Linne (family Tiliaceae), Betula alba Linne (family Betulaceae), Origanum majorana L., Uncaria gambir Roxburgh (family Rubiaceae), Juglans regia Linne var. sinensis De Candolie or other plants of the family Juglandaceae, Sophora flavescens Aiton (family Leguminosae), Sanguisorba officinalis Linne (family Rosaceae), Hypericum perforatum Linne or Hypericum erectum Thunberg (family Guttiferae), Thea sinensis Linne (family Theaceae), Curcuma longa L (family Zingiberaceae), the turmeric purified extract curcumin, Symplocos racemosa, Cyperus rotundus, Cyperus scariosus, Gaultheria fragrantissima, Acacia fornensia, Terminalia chebula, Ficus bengalensis (Banyan tree), Cassia fistula Linn., Lyonia ovalifolia, Calophyllum inophyllum, Ficus religiosa, and the like.

Such plant extracts may be obtained from the roots, leaves, stems, flowers, etc. of herbal plants or from the roots, buds, bark, fruit, leaves, flowers, etc. of woody plants.

The extracts from these plants may be obtained by drying the plant material, if necessary, and further slicing or pulverizing it, if necessary, and then using cold water, warm water or boiling/hot water to prepare aqueous extracts, while organic solvents such as methanol, ethanol, 1,3-butanediol and ether may also be used therewith at ambient temperature or with heating.

Artificial Skin

The basic medium used for production of artificial skin according to the present invention may be any medium which is conventionally used for producting artificial skin, and such media include Dulbecco's modified Eagle medium (DMEM) containing 10% fetal bovine serum; DMEM-Ham's F12 (3:1) containing 10% fetal bovine serum, 5 μg/ml transferrin, 5 μg/ml insulin, 2 nM tri-iodothyronine, 0.1 nM cholera toxin and 0.4 μg/ml hydrocortisone; and a 1:1 mixture of keratinocyte growth medium (KGM) and DMEM containing 10% fetal bovine serum. The amount of serine protease inhibitor added to such basic media will differ depending on the type, but will generally be from about 1 ng/L to 1 g/L. The amount of the matrix protein production accelerator added to the basic medium will be from about 1 ng/L to 1 g/L, and the amount of matrix metalloprotease inhibitor added will be from about 1 nmol/L to 10⁻² mole/L.

For production of artificial skin according to the present invention, human fibroblast-containing contracted Type I collagen gel is first placed on a wire mesh. The human fibroblast-containing contracted Type I collagen gel may be prepared, for example, in the following manner. A fibroblast-suspended collagen solution is prepared while cooling on ice, and then the collagen is gelled in a Petri dish. Next, the gel is peeled from the Petri dish walls and the collagen gel is contracted in a CO₂ incubator.

Epidermal cells, such as normal human epidermal keratinocytes, are then cultured on the collagen gel to form an epidermis. The epidermal layer may be formed by culturing skin cells in the following manner. The contracted collagen gel is placed on a wire mesh and a glass ring is placed over the gel. A human foreskin-derived epidermal keratinocyte suspension is loaded into the glass ring while avoiding leakage. The keratinocytes are allowed to adhere in a CO₂ incubator, and the ring is removed. The medium is filled in up to the border of the epidermal layer, and culturing is continued while exposing the epidermal layer to the air, to form a horny layer.

According to this method it is possible to obtain natural skin-like artificial skin having an adequate layer of basement membrane components deposited between a dermal layer composed of fibroblast-containing Type I contracted collagen gel and an epidermal layer.

Epidermal Basement Membrane Structure Formation Accelerating or Repair Accelerating Preparation

As a result of studying structural changes in the basement membrane of the major cosmetic target, facial skin, based on the reported structural changes of aging characterized by reduplication and disruption of the basement membrane with increasing age (Lavker, J. Invest. Dermatol., 1979, 73, 59-66), it was discovered that such structural changes in the basement membrane begin to occur from the late twenties and accumulate with increasing age (Amano, S. et al., IFSCC Magazine, 4(4), 15-23, 2000). The structural changes in this basement membrane are the skin changes preceding the skin changes that occur with aging, such as wrinkle formation, hardening and loss of elasticity. It is, therefore, believed that it is essential for the expression of normal skin function that the basal keratinocytes be firmly bound to the basement membrane, and that it is important to accelerate repair of the basement membrane. Activity inhibiting wrinkle formation was found using ultraviolet irradiation photoaged mouse models exposed to daily doses of ultraviolet rays. Skin basement membrane regeneration and repair accelerators are thus effective as agents for preventing skin aging.

According to the present invention, therefore, there is newly provided a composition for skin activation or a basement membrane formation accelerating artificial skin culturing solution, which comprises, at a concentration sufficient to exhibit an effect of accelerating epidermal basement membrane regeneration, repair or formation, a mixture of one or more of the aforementioned serine protease inhibitors; a mixture of one or more serine protease inhibitors combined with a mixture of one or more of the aforementioned extracellular matrix protein production accelerators; or a mixture of one or more serine protease inhibitors combined with a mixture of one or more extracellular matrix protein production accelerators and a mixture of one or more of the aforementioned matrix metalloprotease inhibitors. Here, “skin activation” refers to prevention or amelioration of, for example, the loss of skin function that accompanies structural changes in the basement membrane due to aging, and specifically loss of skin elasticity, wrinkles, hardening and the like.

The “concentration sufficient to exhibit an effect of accelerating epidermal basement membrane regeneration, repair or formation” will vary depending on the type of compound used and the other components used in preparation of the composition, as well as the form of preparation and the duration of use.

As mentioned above, the present invention provides, as a preferred mode of the aforementioned skin basement membrane structure formation accelerating preparation, a skin external preparation characterized by comprising an extract derived from a plant belonging to Fagaceae Fagus, 1-acyl lysophospholipid represented by the general formula (1) or (2) above and an extract derived from a plant belonging to Labiatae Mentha. The skin external preparation is preferably used as a skin external preparation for basement membrane protection, as a skin external preparation for rough skin amelioration or as a skin external preparation for prevention of aging.

Plants belonging to Fagaceae Fagus include plants such as Fagus crenata Blume, Fagus japonika Maxim, Fagus grandifolia, Fagus sylvatica L., Fagus sylvatica L. var. pendula, Fagus sylvatica L. var. purpurea or Fagus orientalis Lipsky. The buds, flowers, stems, leaves, fruits, seeds, roots or entirety of such plants may be used. There are no particular restrictions on the method of extraction, and for example, solvents that are commonly used for production of cosmetic materials, like water or a hydrophilic organic solvent such as ethanol, methanol, propanol, butanol or 1,3-butylene glycol, may be used alone or in admixture.

As examples of commercially available Fagus plant extracts for the present invention, there may be mentioned GATULINE™ RC and GATULINE™ R, both commercially manufactured by Gattfosse S. A., France.

Any one or more extracts from these Fagus plants may be selected for use in a skin external preparation according to the mode described above. The content is preferably 0.001-5.0 wt % and more preferably 0.01-1.0 wt % of the total amount of the skin external preparation. If the Fagus plant extract content is less than 0.001 wt %, a sufficient rough skin-ameliorating effect is not obtained, while adding it at greater than 5.0 wt % is uneconomical since no correspondingly greater effect is achieved.

The 1-acyl lysophospholipid may be a commercially available product, or it may be obtained by treating a commercially available phospholipid with phospholipase A2. Alternatively, a synthesized 1,2-diacyl phospholipid may be treated with phospholipase A2 to obtain a 1-acyl lysophospholipid with a constant carbon number. A constant carbon number can also be achieved by reacting up to 1 mole of a fatty acid anhydride or a fatty acid halide with 1 mole of glycerophosphocholine in the presence of a catalyst to obtain lysophosphatidylcholine (Japanese Unexamined Patent Publication (KOKAI) No. 63-225388). Soybean-derived phospholipid or the like may also be treated with phospholipase A2.

In the 1-acyl lysophospholipid, when R¹ is a single acyl group, it is preferably an acyl group derived from an unsaturated fatty acid, and when R² is a single acyl group, it is preferably an acyl group derived from an unsaturated fatty acid, and when R² includes two or more different naturally derived acyl groups, they are preferably soybean-derived fatty acid residue groups.

These may be used alone or in combinations of two or more. Particularly preferred 1-acyl lysophospholipids are those of general formula (2) wherein R² is a fatty acid residue having 18 carbon atoms with 3 unsaturated double bonds, and those of general formula (2) wherein R² is a fatty acid residue having 18 carbon atoms with 1-2 unsaturated double bonds.

As examples of commercially available 1-acyl lysophospholipids, there may be mentioned Lipidure (product of NOF Corp., primarily of general formula (2) where R² is a fatty acid residue having 18 carbon atoms with 2 unsaturated double bonds), San Lysolecithin (product of Taiyo Kagaku Co., Ltd., primarily of general formula (2) where R² is a fatty acid residue having 18 carbon atoms with 2 unsaturated double bonds), and Lysolecithin Kyowa (product of Iwase Cosfa Co., Ltd., primarily of general formula (2) where R² is a fatty acid residue having 18 carbon with 2 unsaturated double bonds).

The content of the 1-acyl lysophospholipid is preferably 0.001-1.0 wt %, and more preferably 0.001-0.1 wt % of the total amount of the skin external preparation. At less than 0.001 wt %, the rough skin-ameliorating effect is reduced, and at greater than 1.0 wt %, discoloration may occur with time, which is undesirable in terms of stability.

The aforementioned extract from a plant belonging to Labiatae Mentha may be obtained by immersing the leaves, stems, flowers or roots of the plant, or a mixture thereof, with an extracting solution or heating to reflux, and then filtering and concentrating. The extracting solvent used for the invention may be any solvent normally used for extraction, and typically an organic solvent, for example, an alcohol such as methanol, ethanol or 1,3-butylene glycol, or aqueous alcohol, acetone, ethyl acetate or the like, either alone or in various combinations. The extract from a plant belonging to Labiatae Mentha according to the present invention may be a commercially available product such as Peppermint Extract (product of Maruzen Pharmaceuticals Co., Ltd.).

The content of the extract from a plant belonging to Labiatae Mentha is preferably 0.001-5.0 wt %, and more preferably 0.001-0.1 wt % of the total amount of the skin external preparation. At less than 0.001 wt % the rough skin-ameliorating effect is reduced, and at greater than 5.0 wt %, discoloration may occur with time, which is undesirable in terms of stability.

The preparation of the present invention may be in the form of an aqueous solution, an oily solution or other type of solution, such as emulsion, cream, gel, suspension, microcapsules, powder, granules, capsules, solid or the like. These may be prepared in their original forms by known methods, or prepared as lotions, emulsions, creams, ointments, plasters, paps, aerosols, injections, oral forms (tablets, powders, granules, pills, syrups, lozenges, etc.), suppositories and the like, and administered to the body by application, attachment, spraying, injection, drinking or insertion. Among such preparation forms, skin external preparations such as lotions, emulsions, creams, ointments, plasters, paps and aerosols are considered to be most appropriate for the preparation according to the invention. The term “skin external preparation” as used herein also includes medicines, quasi drugs and cosmetics.

The preparation of the present invention may also contain as appropriate commonly used excipients and aromas, as well as fats and oils, surfactants, preservatives, metal ion sequestering agents, water-soluble polymers, thickeners, powder constituents, ultraviolet ray protecting agents, humectants, drug agents, antioxidants, pH adjustors, detergents, desiccants, emulsifiers, and the like. When such components are added to the preparation according to the present invention, the addition must be in a range which does not impair the original effect of the invention.

Fats and oils include liquid fats and oils, solid fats and oils, waxes, hydrocarbon oils, higher fatty acids, higher alcohols, synthetic ester oils, silicones and the like.

Specifically there may be mentioned, as liquid oils, avocado oil, camellia oil, primrose oil, turtle oil, macadamia nut oil, corn oil, mink oil, olive oil, rape oil, yolk oil, sesame oil, persic oil, wheat germ oil, sasanqua oil, castor oil, linseed oil, safflower oil, cotton oil, perilla oil, soybean oil, peanut oil, tea seed oil, nutmeg oil, rice bran oil, paulownia oil, kiri oil, jojoba oil, germ oil, triglycerin, glycerin trioctanoate and glycerin triisopalmitate; as solid oils, cacao butter, coconut oil, horse fat, hydrogenated coconut oil, palm oil, beef tallow, mutton tallow, hydrogenated beef tallow, palm kernel oil, lard, beef bone fat, Japan tallow kernel oil, hydrogenated oil, neat's foot oil, Japan tallow and hydrogenated castor oil; as waxes, bees wax, candelilla wax, cotton wax, carnauba wax, bayberry wax, Chinese wax, spermaceti wax, montan wax, bran oil, lanolin, kapok wax, lanolin acetate, liquid lanolin, sugarcane wax, isopropyl lanolin fatty acids, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol and POE hydrogenated lanolin alcohol ether; and as hydrocarbon oils, liquid paraffin, ozokerite, squalane, pristane, paraffin, ceresin, vaseline, and microcrystalline wax.

As higher fatty acids there may be mentioned lauric acid, myristic acid, palmitic acid, stearic acid, behenic acid, oleic acid, 12-hydroxystearic acid, undecylenic acid, isostearic acid, linolic acid, linoleic acid, eicosapentaenoic acid (EPA) and docosahexaenoic acid (DHA).

As higher alcohols, there may be mentioned linear alcohols such as lauryl alcohol, cetyl alcohol, stearyl alcohol, behenyl alcohol, myristyl alcohol, oleyl alcohol and cetostearyl alcohol, or branched alcohols such as monostearyl glycerin ether (batyl alcohol), 2-decyltetradecinol, lanolin alcohol, cholesterol, phytosterol, hexyldodecanol, isostearyl alcohol and octyldodecanol.

As synthetic ester oils, there may be mentioned isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethyleneglycol di-2-ethylhexylate, dipentaerythritol fatty acid ester, N-alkylglycol monoisostearates, neopentylglycol dicaprate, diisostearyl malate, glycerin di-2-heptylundecanoate, trimethylolpropane tri-2-ethylhexylate, trimethylolpropane triisostearate, pentaneerythritol tetra-2-ethylhexylate, glycerin tri-2-ethylhexylate, trimethylolpropane triisostearate, cetyl 2-ethylhexanoate, 2-ethylhexyl palmitate, glycerin trimyristate, tri-2-heptylundecanoic glyceride, castor oil fatty acid methyl ester, oleic acid oil, cetostearyl alcohol, acetoglyceride, 2-heptylundecyl palmitate, diisobutyl adipate, N-lauroyl-L-glutamic-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate and triethyl citrate.

As silicones, there may be mentioned linear polysiloxanes such as dimethylpolysiloxane, methylphenylpolysiloxane and methylhydrogenpolysiloxane, cyclic polysiloxanes such as decamethylpolysiloxane, dodecamethylpolysiloxane and tetramethyltetrahydrogenpolysiloxane, as well as silicone resins and silicone rubbers forming tertiary network structures.

The preparation of the present invention may contain anionic surfactants, cationic surfactants, amphoteric surfactants and non-ionic surfactants, either alone or in combinations.

As examples of anionic surfactants there may be mentioned fatty acid soaps such as soap bases, sodium laurate and sodium palmitate, higher alkylsulfuric acid esters such as sodium lauryl sulfate and potassium lauryl sulfate, alkyl ether sulfuric acid esters such as POE triethanolamine lauryl sulfate and POE sodium lauryl sulfate, N-acyl sarcosinates such as sodium lauroyl sarcosinate, and N-myristoyl-N-methyltaurine sodium, coconut oil fatty acid methyl tauride, and the like, higher fatty acid amide sulfonates such as sodium laurylmethyl tauride, phosphoric acid esters such as POE oleyl ether phosphate sodium and POE stearyl ether phosphate, sulfosuccinate such as sodium di-2-ethylhexylsulfosuccinate, sodium monolauroylmonoethanolamidepolyoxyethylene sulfosuccinate and sodium laurylpolypropyleneglycol sulfosuccinate, alkylbenzene sulfonates such as sodium linear dodecylbenzenesulfonate, triethanolamine linear dodecylbenzenesulfonate and linear dodecylbenzenesulfonic acid, N-acylglutamates such as monosodium N-lauroylglutamate, disodium N-stearoylglutamate and monosodium N-myristoyl-L-glutamate, higher fatty acid ester sulfates such as sodium hydrogenated coconut oil fatty glycerin sulfate, sulfated oils such as turkey red oil, and POE alkyl ether carboxylate, POE alkylallyl ether carboxylate, α-olefin sulfonates, higher fatty ester sulfonates, secondary alcohol sulfuric acid esters, higher fatty acid alkylolamide sulfuric acid esters, sodium lauroylmonoethanolamide succinate, ditriethanolamine N-palmitoyl aspartate and casein sodium.

As examples of cationic surfactants, there may be mentioned alkyltrimethylammonium salts such as stearyltrimethylammonium chloride and lauryltrimethylammonium chloride, alkylpyridinium salts such as distearyldimethylammoniumdialkyldimethylammonium chloride, poly(N,N′-dimethyl-3,5-methylenepiperidinium) chloride and cetylpyridinium chloride, as well as alkyl quaternary ammonium salts, alkyldimethylbenzylammonium salts, alkylisoquinolinium salts, dialkylmorpholinium salts, POE alkylamines, alkylamine salts, polyamine fatty acid derivatives, amyl alcohol fatty acid derivatives, benzalkonium chloride, benzetonium chloride, and the like.

As examples of amphoteric surfactants, there may be mentioned imidazoline-based amphoteric surfactants such as sodium 2-undecyl-N,N,N-(hydroxyethylcarboxymethyl)-2-imidazoline and 1-carboxyethyloxydisodium 2-cocoyl-2-imidazolinium hydroxide, and betaine-based surfactants such as 2-heptadecyl-N-carboxymethyl-N-hydroxyethylimidazolinium betaine, betaine lauryldimethylaminoacetate, alkylbetaines, amidobetaine and sulfobetaine.

As examples of lipophilic non-ionic surfactants, there may be mentioned sorbitan fatty acid esters such as sorbitan monooleate, sorbitan monoisostearate, sorbitan monolaurate, sorbitan monopalmitate, sorbitan monostearate, sorbitan sesquioleate, sorbitan trioleate, diglycerolsorbitan penta-2-ethylhexylate and diglycerolsorbitan tetra-2-ethylhexylate, glycerinpolyglycerin fatty acids such as monocotton oil fatty acid glycerin, glycerin monoerucate, glycerin sesquioleate, glycerin monostearate, glycerin α,α′-oleate pyroglutamate, or malic acid and the like, propylene glycol fatty acid esters such as propylene glycol monostearate, as well as hydrogenated castor oil derivatives, glycerin alkyl ethers, polyoxyethylene methylpolysiloxane copolymers, and the like.

As examples of hydrophilic non-ionic surfactants, there may be mentioned POE sorbitan fatty acid esters such as POE sorbitan monooleate, POE-sorbitan monostearate, POE-sorbitan monooleate and POE-sorbitan tetraoleate, POE sorbitol fatty acid esters such as POE-sorbitol monolaurate, POE-sorbitol monooleate, POE-sorbitol pentaoleate and POE-sorbitol monostearate, POE glycerin fatty acid esters such as POE-glycerin monostearate, POE-glycerin monoisostearate and POE-glycerin triisostearate, POE fatty acid esters such as POE monooleate, POE distearate, POE monodioleate and ethyleneglycol distearate, POE alkyl ethers such as POE lauryl ether, POE oleyl ether, POE stearyl ether, POE behenyl ether, POE 2-octyldodecyl ether and POE cholestanol ether, POE alkylphenyl ethers such as POE octylphenyl ether, POE nonylphenyl ether and POE dinonylphenyl ether, pluronics such as pluronic acid, POE•POP alkyl ethers such as POE•POP cetyl ether, POE•POP 2-decyltetradecyl ether, POE•POP monobutyl ether, POE•POP hydrogenated lanolin and POE•POP glycerin ether, tetra POE•tetra POP ethylenediamine condensates such as tetronics, castor oil and hydrogenated castor oil derivatives such as POE castor oil, POE hydrogenated castor oil, POE hydrogenated castor oil monoisostearate, POE hydrogenated castor oil triisostearate, POE: hydrogenated castor oil monopyroglutamate monoisostearate diester and POE hydrogenated castor oil maleic acid, POE beeswax/lanolin derivatives such as POE sorbitol beeswax, alkanolamides such as coconut oil fatty acid diethanolamide, lauric monoethanolamide and fatty acid isopropanolamides, as well as POE propyleneglycol fatty acid esters, POE alkylamine, POE fatty acid amides, sucrose fatty acid esters, POE nonylphenylformaldehyde condensate, alkylethoxydimethylamine oxides and trioleylphosphoric acid.

As the aforementioned preservatives, there may be mentioned methylparaben, ethylparaben, butylparaben, and the like.

As the aforementioned metal ion sequestering agents, there may be mentioned sodium edetate, EDTA, and the like.

As the aforementioned water-soluble polymers, there may be mentioned natural polymers, semi-synthetic polymers, synthetic polymers and inorganic polymers.

As natural water-soluble polymers, there may be mentioned gum arabic, tragacanth gum, galactan, guar gum, carob gum, karaya gum, carrageenan, tamarind gum, xanthan gum, pectin, agar, quince seed, colloidal algae (seaweed extract), starch (rice, corn, potato, wheat), vegetable polymers such as glycyrrhizinic acid, microorganic polymers such as xanthan gum, dextran, succinoglycan and pullulan, and animal polymers such as collagen, casein, albumin and gelatin.

As semi-synthetic water-soluble polymers, there may be mentioned starch-based polymers such as dextrin, carboxymethyl starch and methylhydroxypropyl starch, cellulose-based polymers such as methyl cellulose, nitrocellulose, ethyl cellulose, methylhydroxypropyl cellulose, hydroxyethyl cellulose, dimethyldialkyl(12-20) ammonium sulfate cellulose, hydroxypropyl cellulose, sodium carboxymethylcellulose (CMC), crystalline cellulose and cellulose powder, and alginic acid-based macromole such as sodium alginate and propyleneglycol alginate.

As synthetic water-soluble polymers, there may be mentioned vinyl-based polymers such as polyvinyl alcohol, polyvinylmethyl ether, polyvinylpyrrolidone, carboxyvinyl polymer and alkyl-modified carboxyvinyl polymer, polyoxyethylene-based polymers such as polyethyleneglycol 2000, 4000 and 6000, polyoxyethylenepolyoxypropylene copolymer-based polymers, acrylic-based polymers such as sodium polyacrylate, polyethylene acrylate and polyacrylamide, as well as polyethyleneimine, cationic polymers, and the like.

As inorganic water-soluble polymers, there may be mentioned bentonite, aluminum magnesium silicate, laponite, hectorite, silicic anhydride, and the like.

As the aforementioned powder constituents, there may be mentioned inorganic powders such as talc, kaolin, mica, sericite, muscovite, bronze mica, synthetic mica, lepidolite, black mica, lithia mica, vermiculite, magnesium carbonate, calcium carbonate, aluminum silicate, barium silicate, calcium silicate, magnesium silicate, strontium silicate, metal tungstate salts, magnesium, silica, zeolite, barium sulfate, calcined calcium sulfate (calcined gypsum), calcium phosphate, fluoroapatite, hydroxyapatite, ceramic powder, metallic soap (zinc myristate, calcium palmitate, aluminum stearate) and boron nitride, organic powders such as polyamide resin powders (nylon powders), polyethylene powder, polymethyl methacrylate powder, polystyrene powder, styrene/acrylic acid copolymer resin powder, benzoguanamine resin powder, polytetrafluoroethylene powder and cellulose powder, inorganic white pigments such as titanium dioxide and zinc oxide, inorganic red pigments such as iron oxide (bengala) and iron titanate, inorganic brown pigments such as γ-iron oxide, inorganic yellow pigments such as yellow iron oxide and loess, inorganic black pigments such as black iron oxide, carbon black and lower titanium oxide, inorganic purple pigments such as mango violet and cobalt violet, inorganic green pigments such as chromium oxide, chromium hydroxide and cobalt titanate, inorganic blue pigments such as ultramarine and Prussian blue, pearl pigments such as titanium oxide-coated mica, titanium oxide-coated bismuth oxychloride, titanium oxide-coated talc, colored titanium oxide-coated mica, bismuth oxychloride and pearl essence, metallic powder pigments such as aluminum powder and copper powder, organic pigments of zirconium, barium and aluminum lake such as Red #201, Red #202, Red #204, Red #205, Red #220, Red #226, Red #228, Red #405, Orange #203, Orange #204, Yellow #205, Yellow #401, Yellow #404, Red #3, Red #104, Red #106, Red #227, Red #230, Red #401, Red #505, Orange #205, Yellow #4, Yellow #5, Yellow #202, Yellow #203, Green #3 and Blue #1, natural pigments such as chlorophyll and β-carotene, or titanium yellow, carthamin, safflower red, and the like.

The aforementioned ultraviolet ray protecting agents include both substances that chemically absorb ultraviolet rays, i.e. “ultraviolet absorbers”, and substances that disperse or reflect ultraviolet rays by physical action, i.e. “ultraviolet blockers”.

As long-wavelength ultraviolet ray (UVA) absorbers, there may be mentioned anthranilic acid-based ultraviolet absorbers such as methylanthranilate and homomenthyl-N-acetylanthranilate, benzophenone-based ultraviolet absorbers such as 2,4-dihydroxybenzophenone, 2,2-dihydroxy-4-methoxybenzophenone, 2,2′-dihydroxy-4,4′-dimethoxybenzophenone, 2,2′,4,4′-tetrahydroxybenzophenone,-2-hydroxy-4-methoxybenzophenone and 2-hydroxy-4-methoxy-4′-methylbenzophenone, 2-hydroxy-4-methoxybenzophenone-5-sulfone hydrochloride, 4-phenylbenzophenone, 2-ethylhexyl-4′-phenylbenzophenone-2-carboxylate, 2-hydroxy-4-n-octoxybenzophenone and 4-hydroxy-3-carboxybenzophenone, benzotriazole-based ultraviolet absorbers such as 2,2′-hydroxy-5-methylphenylbenzotriazole, 2-(2′-hydroxy-5′-t-octylphenyl)benzotriazole and 2-(2′-hydroxy-5═-methylphenyl)benzotriazole, as well as dianisoylmethane, 4-methoxy-4′-t-butyldibenzoylmethane, and the like.

Among these long-wavelength ultraviolet ray absorbers, 4-methoxy-4′-tert-butyldibenzoylmethane, 2-hydroxy-4-methoxybenzophenone and 2-hydroxy-4-methoxybenzophenone derivatives such as 2-hydroxy-4-methoxybenzophenone-5-sulfone hydrochloride are preferred for their excellent safe and efficient properties.

As medium-wavelength ultraviolet ray (UVB) absorbers there may be mentioned benzoic acid-based ultraviolet absorbers such as para-aminobenzoic acid (hereinafter referred to as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester and N,N-dimethyl PABA amyl ester, salicylic acid-based ultraviolet absorbers such as dipropyleneglycol salicylate, ethyleneglycol salicylate, myristyl salicylate, methyl salicylate, amyl salicylate, menthyl salicylate, homomenthyl salicylate, octyl salicylate, phenyl salicylate, benzyl salicylate and p-isopropanolphenyl salicylate, cinnamic acid-based ultraviolet absorbers such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate (2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate and glyceryl mono-2-ethylhexanoyl-diparamethoxy cinnamate, octyl methoxycinnamate, 3-methyl-4-[methylbis(trimethylsiloxy)silyl]butyl 3,4,5-trimethoxy cinnamate and monoethyl p-dimethoxycinnamate, camphor derivatives such as 3-(4′-methylbenzylidene)-d,1-camphor, 3-benzylidene-d,1-camphor and 5-(3,3-dimethyl-2-norbornylidene)-3-pentyn-2-one, as well as urocanic acid, ethyl urocanate, 2-phenyl-5-methylbenzoxazole, dibenzalazine, and the like.

As ultraviolet blockers there may be mentioned titanium oxide (TiO₂)m talc (MgSiO₂), carmine (FeO₂), bentonite, kaolin, zinc oxide (ZnO), and the like.

As examples of humectants there may be mentioned polyethylene glycol, polypropylene glycol, glycerin, 1,3-butylene glycol, hexylene glycol, xylitol, sorbitol, maltitol, chondroitin sulfate, hyaluronic acid, mucoitin sulfate, charonin acid, atelocollagen, cholesteryl-12-hydroxystearate, sodium lactate, bile salts, dl-pyrrolidone carboxylic acid, short-chain soluble collagen, diglycerin (EO) PO addition product, chestnut rose extract, yarrow extract, melilot extract, and the like.

As drug agents, there may be mentioned arbutin, vitamin C and its derivatives, kojic acid, placental extract, glutathione, whiteners such as geranium extract, glycyrrhizinic acid derivatives, glycyrrhetic acid derivatives, salicylic acid derivatives, hinokitiol, zinc oxide, antiphlogistic agents such as allantoin, royal jelly, photosensitive elements, cholesterol derivatives, activating agents such as young calf hemolysed blood extract, nonylic acid vanillylamide, benzyl nicotinate, β-butoxyethyl nicotinate, capsaicin, zingherone, cantharis tincture, ichthamol, caffeine, tannic acid, α-borneol, tocopherol nicotinate, inositol hexanicotinate, cyclandelate, cinnarizine, tolazoline, acetylcholine, blood circulation promoters such as verapamil, cepharanthin and γ-orizanol or antiseborrheic agents such as sulfur and thiantol; and for various purposes, cork tree bark extract components, coptis extract components, lithospermum extract components, peony extract components, swertia herb extract components, birch extract components, sage extract components, loquat extract components, carrot extract components, aloe extract components, mallow extract components, iris extract components, grape extract components, coix seed extract components, loofah extract components, lily extract components, saffron extract components, cnidium root extract components, ginger extract components, St. John's wort extract components, cammock extract components, rosemary extract components, garlic extract components, capsicum extract components, citrus peel, angelic root, and the like, A vitamins such as retinol and retinol acetate, B₂ vitamins such as riboflavin, riboflavin butyrate and flavin adenine nucleotide, B₆ vitamins such as pyridoxine hydrochloride and pyridoxine dioctanoate, C vitamins such as L-ascorbic acid, L-ascorbyl dipalmitate, sodium L-ascorbyl 2-sulfate, L-ascorbyl phosphate and dipotassium DL-α-tocopherol-L-ascorbyl phosphate diester, pantothenic acid derivatives such as calcium pantothenate, D-pantothenyl alcohol, pantothenyl ethyl ether and acetylpantothenyl ethyl ether, D vitamins such as ergocalciferol and cholecalciferol, nicotinic acid derivatives such as nicotinic acid, nicotinamide and benzyl nicotinate, E vitamins such as α-tocopherol, tocopherol acetate, DL-α-tocopherol nicotinate and DL-α-tocopherol succinate, and other vitamins such as vitamin P, biotin and the like.

These drug agents may be added in a wide range so long as their addition does not impair the intended effect of the invention.

The preparation of the present invention prepared in this manner can prevent loss of function of the basement membrane that occurs with its structural changes, and promote skin activation.

### EXAMPLES

The present invention will now be explained in further detail through examples.

### Example 1

### Production of Artificial Skin using Matrix Metalloprotease Inhibitors, Extracellular Matrix Production Accelerators (Cytokines) and Serine Protease Inhibitors

Collagen gel was obtained by preparing 10 ml of a human dermal fibroblasts (0.3-1×10⁵ cell/ml)-suspended collagen solution (using I-AC collagen manufactured by Koken Co., Ltd.) on ice, followed by gelling the collagen in a 60 mm Petri dish at 37° C. The gel was then peeled from the bottom of the dish, the collagen gel was placed on metal, and a glass ring (12 mm inner diameter) was placed over the gel. A 0.4 ml portion of a human foreskin-derived epidermal keratinocyte (1×10⁶/ml) suspension (KGM-DMEM (1:1) mixed medium containing 5% fetal bovine serum) was poured into the glass ring while avoiding leakage. The keratinocytes were allowed to adhere in a CO₂ incubator overnight, and the ring was removed on the following day. The medium was filled in up to the border of the epidermal layer, and culturing was continued while exposing the epidermal layer to the air, to prepare a multilayered skin model with epidermis having a horny layer.

From the 4th day after seeding the epidermal cells, the medium was replaced with medium containing (1) no compound (−compound A), (2) 10 uM of compound A (+compound A), (3) 10 μM of compound A and 10 μg/ml of aprotinin (+compound A+aprotinin), (4) 10 μM of compound A and 1 ng/ml of interleukin-1β (+compound A+IL-1), (5) 10 μM of compound A, 1 ng/ml of interleukin-1β and 10 βg/ml of aprotinin (+compound A+IL-1+aprotinin), (6) 10 μM of compound A and 1 ng/ml of TFG-α (+compound A+TGFα), (7) 10 μM of compound A, 1 ng/ml of TGF-α and 10 μg/ml aprotinin (+compound A, +TGFα+ aprotinin), (8) 10 μM of compound A and 100 ng/ml PDGF (+compound A+PDGF) and (9) 10 μm of compound A, 100 ng/ml PDGF and 10 μg/ml of aprotinin (+compound A+PDGF+aprotinin), and every 2-3 days thereafter, the medium was exchanged with a medium containing the same types and same concentrations of matrix metalloprotease, extracellular matrix production accelerator (cytokines) and serine protease inhibitor, after which culturing was performed for 2 weeks.

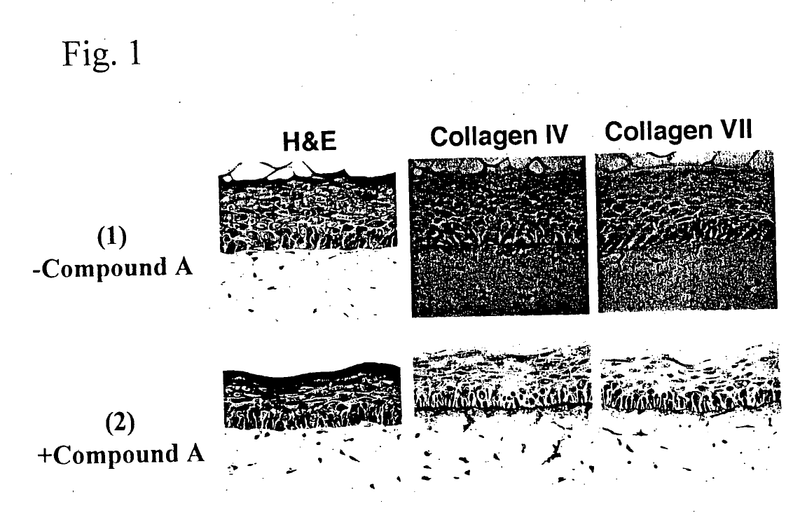

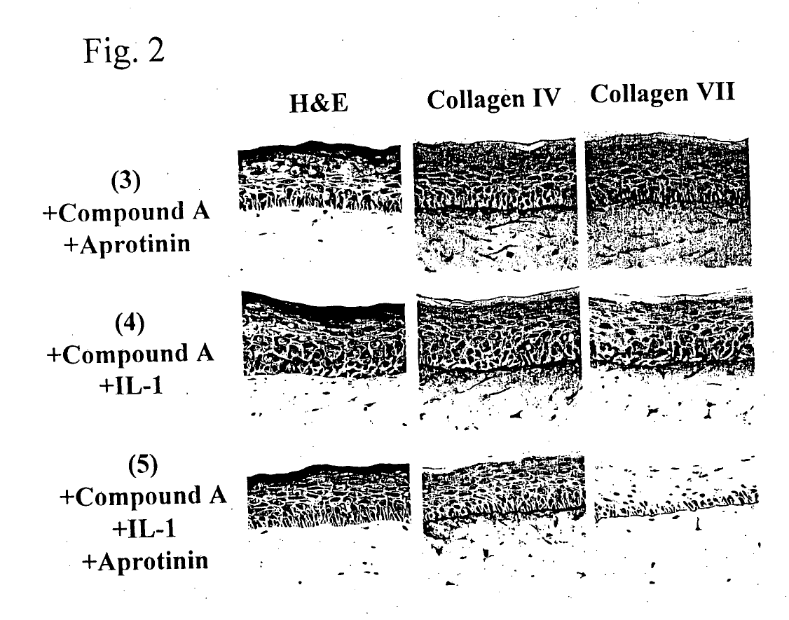

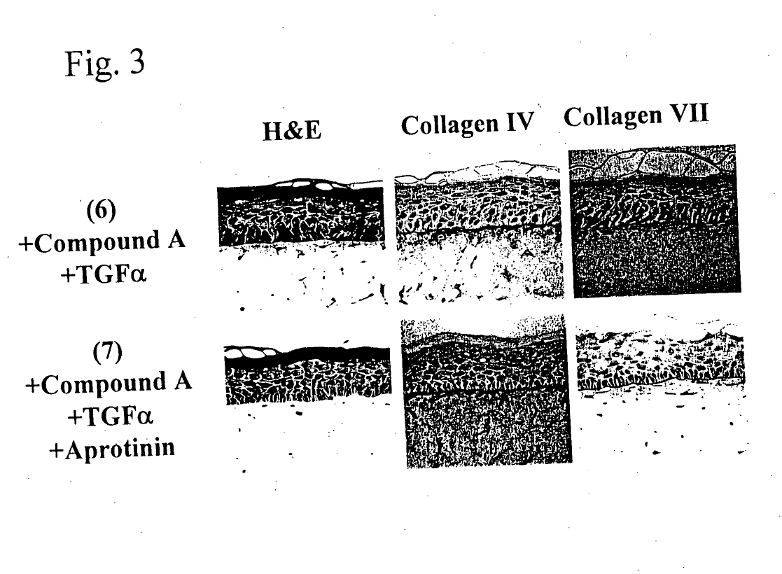

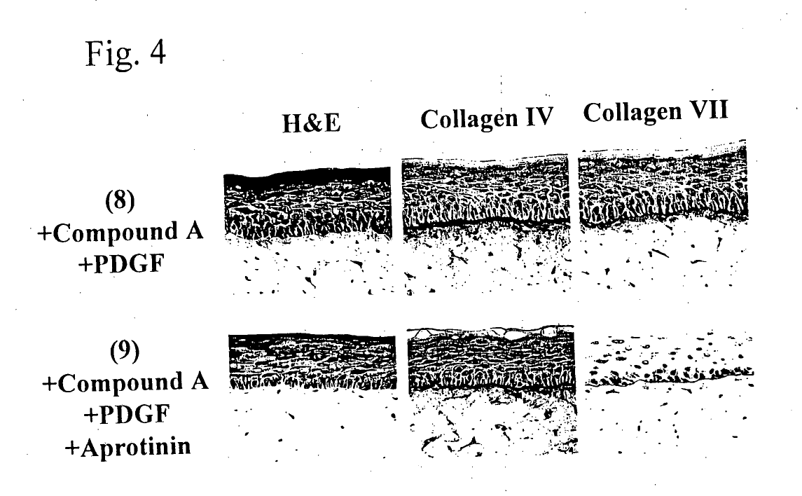

The formed artificial skin was stained with hematoxylin and eosin (H&E) and immunostaining (using anti-type IV collagen antibody and anti-type VII collagen antibody). The results are shown in FIGS. 1 to 4.

Although virtually no staining of the type VII collagen directly under the basal keratinocytes was observed in the control (1), weak but definite staining of type VII collagen was observed when the matrix metalloprotease inhibitor compound A (2) was added. The staining of type VII collagen further increased when the serine protease inhibitor aprotinin (3) was added in addition to the matrix metalloprotease inhibitor compound A. Staining of type VII collagen was particularly accentuated when compound A was accompanied with the matrix protein production-increasing compounds interleukin 1 (IL-1) (4), transforming growth factor-α (TGF-α) (6) and platelet-derived growth factor (PDGF) (8). When the matrix protein production-increasing compounds interleukin 1 (IL-1) (5), transforming growth factor-α (TGF-α) (7) and platelet-derived growth factor (PDGF) (9) were combined with aprotinin and compound A, the staining of type IV and type VII collagen was clearly concentrated directly under the basal keratinocytes, giving a sharp stained image, thus suggesting that each of the molecules probably constructs an appropriate three-dimensional structure.

The same culturing process as described above was carried out using the following plant extracts instead of the aforementioned matrix metalloprotease inhibitor, compound A.

Thymus serpyllum L., Valeriana fauriei Briquet and other plants of the family Valerianaceae, Diospyros kaki Thunberg (family Ebenaceae), Astragalus sinicus Linne (family Leguminosae), Crataegus cuneata Siebold et Zuccarini (family Rosaceae), Paeonia suffruticosa Andrews (Poeonia montan Sims) (family Paconiaceae), Thea sinensis Linne var. assamica Pierre, (family Theaceae), Eucalyptus globulus Labillardiere and other plants of the family Myrtaceae, Potentilla tormentilla Schrank (family Rosaceae), Tilia cordata Mill., Tilia platyphyllus Scop., Tilia europaea Linne (family Tiliaceae), Betula alba Linne (family Betulaceae), Origanum majorana L., Uncaria gambir Roxburgh (family Rubiaceae), Juglans regia Linne var. sinensis De Candolie or other plants of the family Juglandaceae, Sophora flavescens Aiton (family Leguminosae), Sanguisorba officinalis Linne (family Rosaceae), Hypericum perforatum Linne or Hypericum erectum Thunberg (family Guttiferae), Thea sinensis Linne (family Theaceae), Curcuma longa L (family Zingiberaceae), the turmeric purified extract curcumin, Symplocos racemosa, Cyperus rotundus, Cyperus scariosus, Gaultheria fragrantissima, Acacia fornensia, Terminalia chebula, Ficus bengalensis (Banyan tree), Cassia fistula Linn., Lyonia ovalifolia, Calophyllum inophyllum, Ficus religiosa.

The similar results were obtained as when compound A was used as the matrix metalloprotease.

The same culturing process as described above was carried out using the following plant extracts instead of the aforementioned serine protease inhibitor, aprotinin.

Calophyllum brasiliense Cambess., Myrcia sphaerocarpa DC, Hyptis crenata Pohl ex Benth., C. rotundus L, E. sylvestris var. ellipticus, E. decipiens Helmsl., plants belonging to genera Rosa, Rubus, Filipendula and Crataegus of the Rosaceae family; plants belonging to the family Paeoniaceae, genus Paeonia; plants belonging to the family Moraceae, genus Humulus, plants belonging to the family Compositae, genus Arnica and the family Compositae, genus Anthemis, plants belonging to the family Pyrolaceae, plants belonging to the family Hypericaceae; plants belonging to Mentha piperata L. and Mentha viridis L.; plants belonging to Compositae Carthamus tinctorius L., Geraniaceae Geranium thunbergii Siebold et Zuccarini and Vitaceae Vitis; plants belonging to the family Betulaceae such as Betula alba L., Betula lutea L. and Betula pendula Roth.; plants belonging to the family Rosaceae, genus Prunus, which are apricots (P. armeniaca L., P ansu Komar, P. mandshurica Koehne, P. sibirica L.); and zapote, que shar, sano-sano, cora de cavallo.

The similar results were obtained as when aprotinin was used as the serine protease inhibitor.

The same culturing process as described above was carried out using, instead of the matrix protein production accelerators IL-1, TGF-α and PDGF, the following laminin 5 production-accelerating agents: extracts of soybean lysolecithin, glycyrrhiza, blackberry lily, jitanoki, Tinospora crispa, fenugreek, Papaveraceae Bocconia palo amarillo, Leguminosae Psophocarpus tetragonolobus, Leguminosae Cassia retama, Gentianaceae Erythraea chilensis (canchalagua), soybean, pueraria root, cammock, melilot, sprouts and red bean. The same culturing was also carried out using, as type IV collagen or type VII collagen production-accelerating agents, pule (Alstonia scholaris), beech (Fagus sylvatica), kudzu vine Pueraria lobata Ohwi), and English ivy (Hedera helix) extracts, and as a type VII collagen production-accelerating agent, orchid extract. The similar results were obtained as when IL-1, TGF-α and PDGF were used.

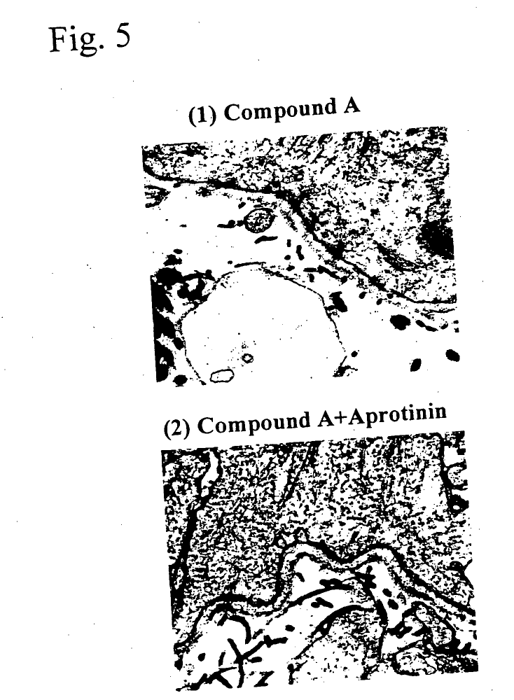

A transmission electron microscope was used to observe the basement membrane structure directly under the epidermis after adding the matrix metalloprotease inhibitor compound A (2) or after adding the serine protease inhibitor aprotinin (3) with the matrix metalloprotease inhibitor compound A, and as shown in FIG. 5, compound A induced formation of the basement membrane, while further addition of aprotinin increased the electron density and accentuated the continuity of the basement membrane.

### Example 2

### Production of Artificial Skin using Extracellular Matrix Production Accelerators and Serine Protease Inhibitors

The method of Example 1 was repeated, but the culturing process was conducted with the following test substances.
(1) No addition (control);
(2) 30 μg/ml of the laminin 5 production accelerator “soybean lysolecithin” alone (soybean LPC);
(3) 0.5% of the serine protease inhibitor “peppermint extract” alone;
(4) 0.3% of the type IV and type VII collagen production accelerator “beech bud extract” alone;
(5) 30 μg/ml of the laminin 5 production accelerator “soybean lysolecithin”, 0.5% of the serine protease inhibitor “peppermint extract” and 0.3% of the type IV and type VII collagen production accelerator “beech bud extract” (mixture).

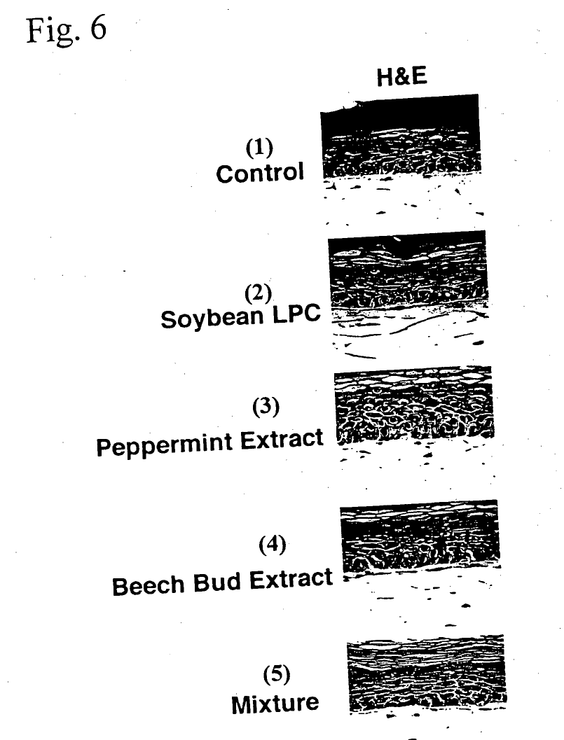

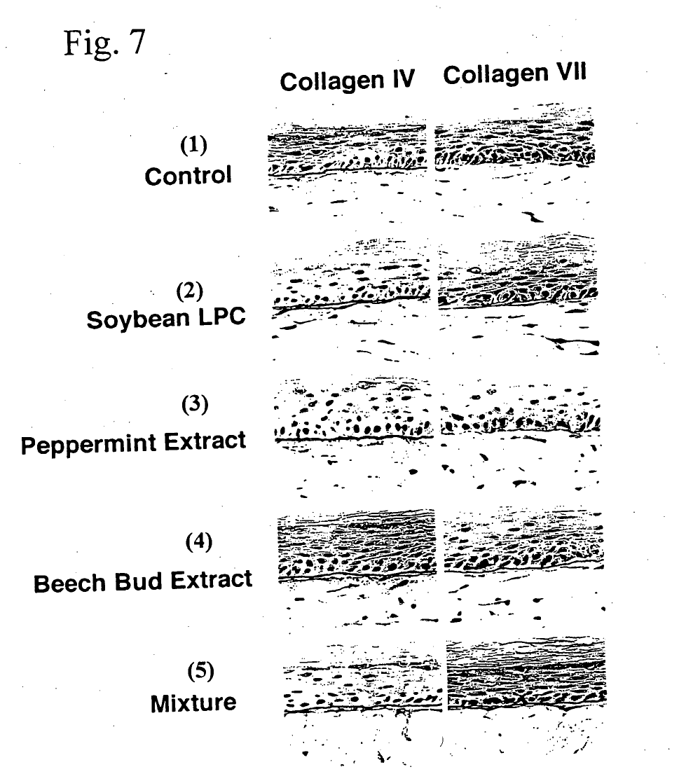

The formed artificial skin was stained with hematoxylin and eosin (H&E) staining and immunostaining (using anti-type IV collagen antibody and anti-type VII collagen antibody). The results are shown in FIGS. 6 and 7.

In the control (1), the type IV collagen was weakly stained but virtually no staining of the type VII collagen was observed. In contrast, the staining of type IV collagen increased with the laminin 5 production accelerator, soybean lysolecithin (2). The staining of type VII collagen increased with the peppermint extract (3). The production of type IV and type VII collagen increased with the beech bud extract (4). By combining them (5), a clear increase was confirmed in the staining of type VII collagen.

### Example 3

Skin care effect of the preparation comprising beech bud extract, peppermint extract and soybean lysolecithin (Lipidure) was evaluated by applying it to the skins of three healthy men who were at least 30 years old. As an indication of the effect, horny layer moisture was measured using Corneometer CM 825C (Courage and Khazaka Electric GmbH) according to the instruction supplied by the manufacture. The test preparation was prepared by combining equivalent amounts of beech bud extract and peppermint extract, followed by adding Lipidure to an amount of 0.001 wt %. Ion exchanged water was used as the control. 18 μl of the preparation was applied to each subject at the inside portion of their forearm in an area of 3 square cm. After one and two hours following the application, the horny layer moisture was measured. The results are shown in the following table.

TABLE 1
Increasing Effect on Horny Layer Moisture

| Increase Ratio | Control Site | Preparation applied site |
|---|---|---|
| 1 hr later | −2.5% | 44.4% (p < 0.01) |
| 2 hr later | 1.0% | 12.1% (p < 0.01) |

The results in Table 1 showing the effect of improving the horny layer moisture indicates that application of the test preparation results in increase of the moisture content in the horny layer, whereby exhibits a skin ameliorating effect.

### Example 4

Cream

A. Oily Phase

| Nylon powder | 1.0 | wt % |
|---|---|---|
| Cyclomethicone | 20.0 | |
| Squalane | 2.0 | |
| Cetyl octanoate | 5.0 | |
| Dimethicone | 1.0 | |
| Paraben | q.s. | |
| Aromatics | q.s. | |
| Isostearic acid | 1.0 | |
| Dimethyldistearylammonium hectorite | 2.0 | |

B. Aqueous Phase

| | |
|---|---|
| Polyoxyalkylene-modified organopolysiloxane | 2.0 |
| Ion-exchange water | remainder |
| Butylene glycol | 7.0 |
| Dipropylene glycol | 7.0 |
| Edetate | q.s. |
| Polyethylene glycol | 1.0 |
| Phenoxyethanol | q.s. |
| Pearl extract | q.s. |
| Beech bud extract | 1.0 |
| Soybean lysolecithin (Lipidure) | 0.1 |
| Peppermint extract | 1.0 |

(Preparation method) Phase A and phase B were each heated at 70° C. to thorough dissolution. Phase A was added to phase B and emulsified with an emulsifier. The emulsion was cooled with a heat-exchanger to obtain a cream. Rough skin preventing and rough skin ameliorating effect test

The cream with the formulation of Example 3 above was used for a rough skin preventing and rough skin ameliorating effect test with female subjects aged 28 to 65. Specifically, the cream of Example 3 was applied daily to the faces of healthy women for a period of 4 weeks. The condition of the skin was observed by the following method. The transepidermal water loss was measured with a Tewameter TM210 (Courage and Khazaka Electric GmbH) according to the instruction supplied by the manufacturer, and the horny layer moisture was measured using a Corneometer CM 825C (Courage and Khazaka Electric GmbH) according to the instruction supplied by the manufacturer. The skin surface replica analysis was performed by taking a replica of the skin surface using a slide glass smeared with cyanoacrylate glue (Cyanolit, product of Eleco) and using an optical microscope (OLYMPUS SZ 4045 TR) to visually observe the condition of the skin surface formed by grooves and ridges.

TABLE 2
Amelioration (Reduction) of Transepidermal Water Loss
with 4-week Application

| Amelioration rate | Control site* | Cream-applied site |
|---|---|---|
| | 15% | 27% (p < 0.002) |

*No cream was applied to the control site

TABLE 3
Amelioration (Increase) of Horny Layer Moisture
with 4-week Application

| Amelioration rate | Control site* | Cream-applied site |
|---|---|---|
| | −1% | 22% (p < 0.001) |

*No cream was applied to the control site

TABLE 4
Amelioration of Skin Surface Replica with 4-week Application

| | |
|---|---|
| Amelioration rate with cream application | 43% (p < 0.002) |

The results in Table 2 showing the transepidermal water loss ameliorating effects indicate that continuous application of the cream for 4 weeks improved the skin, restored the barrier effect and reduced moisture perspiration from the epidermis.

The results in Table 3 showing the horny layer moisture ameliorating effects indicate that continuous application of the cream for 4 weeks improved the skin, increased the moisture in the horny layer and enhanced the skin condition.

Also, the results in Table 4 showing the skin surface texture ameliorating effects indicate that continuous application of the cream for 4 weeks improved the skin, ameliorated the surface texture of the skin composed of grooves and ridges, and generally enhanced the skin condition.

Formulation examples of skin external preparations of the present invention in various forms will now be explained.

### Example 5

Cream

A. Oily Phase

| | | |
|---|---|---|
| Stearic acid | 10.0 | wt % |
| Stearyl alcohol | 5.0 | |
| Butyl stearate | 10.0 | |
| Monoglycerin stearate | 2.0 | |
| Vitamin E acetate | 0.5 | |
| Vitamin A palmitate | 0.1 | |
| Macadamia nut oil | 1.0 | |

| | |
|---|---|
| Aromatics | q.s. |
| Preservatives | q.s. |

B. Aqueous Phase

| | |
|---|---|
| Glycerin | 5.0 |
| 1,2-pentanediol | 3.0 |
| Acetylated hyaluronic acid | 2.0 |
| Potassium hydroxide | 0.5 |
| Magnesium phosphate ascorbate | 0.5 |
| Tranexamic acid | 0.1 |
| Trisodium edetate | q.s. |
| Beech bud extract | 1.0 |
| Soybean lysolecithin (Lipidure) | 0.1 |
| Peppermint extract | 1.0 |
| Purified water | q.s. |

(Preparation method) The oily phase A and aqueous phase B were each heated at 70° C. to thorough dissolution. Phase A was added to phase B and emulsified with an emulsifier. The emulsion was cooled with a heat-exchanger to obtain a cream.

### Example 6

Cream

A. Oily Phase

| | | |
|---|---|---|
| Cetanol | 4.0 | wt % |
| Vaseline | 7.0 | |
| Isopropyl myristate | 8.0 | |
| Squalane | 15.0 | |
| Monoglycerin stearate | 2.2 | |
| POE(20)′ sorbitan monostearate | 2.8 | |
| Vitamin E nicotinate | 2.0 | |
| Aromatics | 0.3 | |

| | |
|---|---|
| Antioxidants | q.s. |
| Preservatives | q.s. |

B. Aqueous Phase

| | |
|---|---|
| Glycerin | 10.0 |
| Dipropylene glycol | 4.0 |
| Sodium pyrrolidonecarboxylate | 1.0 |
| Beech bud extract | 1.0 |
| Soybean lysolecithin (Lipidure) | 0.1 |
| Peppermint extract | 1.0 |
| Disodium edetate | q.s. |
| Purified water | remainder |

(Preparation method) The cream was obtained in the same manner as Example 4.

### Example 7

Emulsion

A. Oily Phase

| | | |
|---|---|---|
| Squalane | 5.0 | wt % |
| Oleyl oleate | 3.0 | |
| Vaseline | 2.0 | |
| Sorbitan sesquioleate | 1.0 | |
| Polyoxyethylene oleyl ether (20 EO) | 1.5 | |
| Primrose oil | 0.5 | |
| Aromatics | 0.5 | |

| | |
|---|---|
| Preservatives | q.s. |

B. Aqueous Phase

| | |
|---|---|
| 1,3-butyleneglycol | 5.0 |
| Melissa extract | 2.0 |
| Ethanol | 3.0 |
| Carboxyvinyl polymer | 0.5 |
| Potassium hydroxide | 0.5 |
| Beech bud extract | 1.0 |
| Soybean lysolecithin (Lipidure) | 0.1 |
| Peppermint extract | 1.0 |
| Sodium hexametaphosphate | 0.05 |
| Purified water | remainder |

(Preparation method) An emulsion was obtained according to the method of Example 5.

### Example 8

Foundation

A. Oily Phase

| | |
|---|---|
| Cetanol | 3.0% |
| Deodorant lanolin | 4.0 |
| Jojoba oil | 5.0 |
| Vaseline | 5.0 |
| Squalane | 6.0 |
| Monoglycerin stearate | 3.0 |
| POE(60) hydrogenated castor oil | 2.0 |
| POE(20) cetyl ether | 1.0 |
| Pyridoxine tripalmitate | 0.1 |
| Preservatives | q.s. |
| Aromatics | 0.5 |

B. Aqueous Phase

| | |
|---|---|
| Propylene glycol | 10.0 |
| Beech bud extract | 1.0 |
| Soybean lysolecithin (Lipidure) | 0.1 |
| Peppermint extract | 1.0 |
| Trisodium edetate | q.s. |
| Purified water | remainder |

(Preparation method) A foundation was obtained according to the method of Example 5.

### Example 9

Cosmetic Water

A. Oily Phase

| | | |
|---|---|---|
| Ethanol | 5.0 | wt % |
| POE oleyl alcohol ether | 2.0 | |
| 2-ethylhexyl-p-dimethylaminobenzoate | 0.2 | |

| | |
|---|---|
| Preservatives | q.s. |
| Aromatics | q.s. |

B. Aqueous Phase

| | |
|---|---|
| 1,3-butyleneglycol | 9.5 |
| Sodium pyrrolidonecarboxylate | 0.5 |
| Nicotinic amide | 0.5 |
| Glycerin | 5.0 |
| Hydroxypropyl β-cyclodextrin | 1.0 |
| Citric acid | 0.1 |
| Sodium citrate | 0.1 |
| Beech bud extract | 1.0 |
| Soybean lysolecithin (Lipidure) | 0.1 |
| Peppermint extract | 1.0 |
| Purified water | remainder |

(Preparation method) The alcohol phase A was added to the aqueous phase B and solubilized to obtain cosmetic water.

### Example 10

Pack

| | | |
|---|---|---|
| (1) Polyvinyl alcohol | 10.0 | wt % |
| (2) Polyethyleneglycol (molecular weight: 400) | 0.4 | |
| (3) Glycerin | 3.0 | |
| (4) Ethanol (95%) | 8.0 | |
| (5) Beech bud extract | 1.0 | |
| (6) Soybean lysolecithin (Lipidure) | 0.1 | |
| (7) Peppermint extract | 1.0 | |

| | |
|---|---|
| (8) Preservatives | q.s. |
| (9) Aromatics | q.s. |
| (10) Purified water | remainder |

(Preparation method) Components (4), (8) and (9) were mixed, and this mixture was added to a combined solution of (1), (2), (3) and (5), (6), (7) and (10) at 80° C. with stirring, after which the mixture was cooled to room temperature to obtain a pack.

As explained above, a skin external preparation according to the present invention has an excellent basement membrane-ameliorating effect and an excellent effect of improvement and prevention of various skin diseases and rough skin, while also exhibiting action of preventing skin aging through amelioration of the skin.

### Example 11

Artificial Skin Transplant

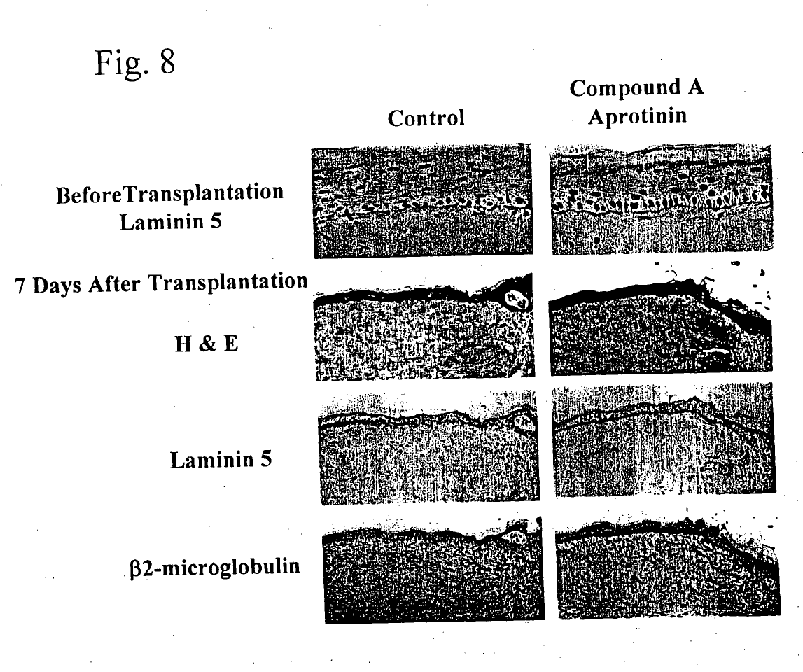

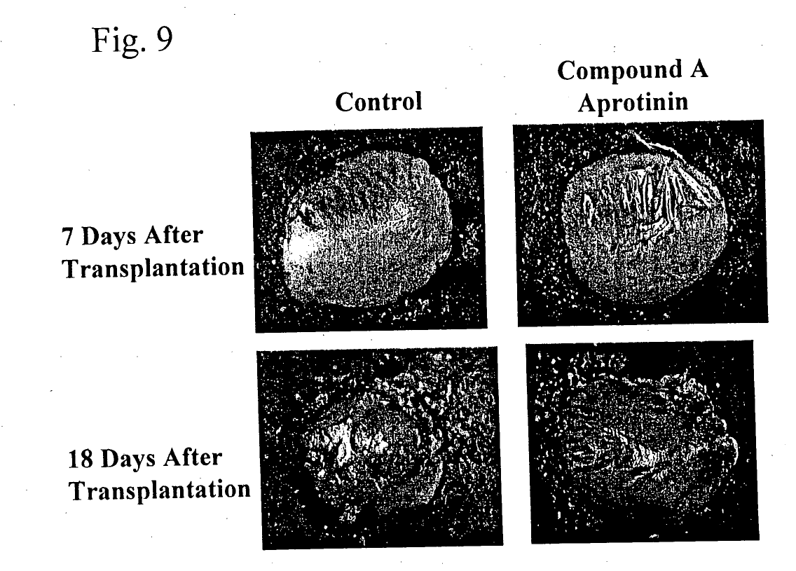

Artificial skin was prepared in the same manner as Example 1. Following the loading of epidermal cells, the artificial skins were cultured from three days to one week, in the absence of any additive, i.e. the control artificial skin, or in the presence of the basement membrane ameliorator, matrix metalloprotease inhibitor compound A (10 μl) and the serine protease inhibitor aprotinin (10 μg/ml), after which each of them was transplanted at dorsal skin-resected sites of nude mice. Silicone gauze was placed over each transplant site, and the site was covered with Tegaterm elastic plastic adhesive tape (Sumitomo 3M). 7 days after the transplantation, external observation was carried out, and then the tissues were collected. The collected tissues were fixed with cold acetone, and paraffin sections were prepared in a conventional way. The transplanted artificial skin was stained with hematoxylin and eosin staining (H&E) or immunostained with anti-laminin 5 antibody (laminin 5). The results are shown in FIGS. 8 and 9.

Referring to FIG. 8, it can be seen that, by comparing the transplanted artificial skins, in the control group, some of the laminin 5 regions underneath the epidermis are remained unstained, while in the group cultured in the presence of matrix metalloprotease inhibitor compound A and aprotinin, a better and mostly uniformed laminin 5 staining was resulted. A histological analysis carried out at 7 days after the transplantation showed that, in the control group, gaps at the dermal epidermal junction region in the artificial skin were observed, and it was also found that the stained laminin 5 region was broadened, suggesting a possibility of the damage of the basement membrane. Further, the thickness and the configuration of the epidermis were being inferior. On the contrary, in the additive group, the configuration of the dermal-epidermal junction region was good, and it also showed a continuous staining of laminin 5. Further, the state of the epidermis was better.

Moreover, this difference is distinct when carrying out an observation of the boundary portion between the artificial skin and the true skin. In particular, when transplanting the artificial skin having a satisfactory basement membrane, in comparison with the control group, it could be seen that the transplanted artificial skin shows a good adhesion, and the staining of the human-derived laminin 5 and the staining of the β-microglobulin, which specifically recognizes a human cell, extremely close to the boundary were observed, and the satisfactory skin prevented the penetration of the mouse-derived epidermis. Also, an angiogenesis originated from the boundary portion was promoted. Thus, if transplanting an artificial skin having a satisfactory basement membrane due to the incorporation of the matrix metalloprotease inhibitor and aprotinin, it could be seen that the post-transplantation state would significantly improved.

Comparing the external photographs (FIG. 9), with respect to the state at day 18 after the transplantation, in the artificial skin group having a satisfactory basement membrane due to the incorporation of the matrix metalloprotease inhibitor and aprotinin, in comparison with the additive-free control group, the contraction caused by the surrounding mouse skin was suppressed, and the formation of the surface horny layer was also significantly improved.

It was thus confirmed that transplantation of skin models with satisfactory basement membranes results in a good adhesiveness of the transplanted tissue, as well as a more attractive maintenance of the transplant sites due to a more satisfactory epidermal and dermal condition.

Serine protease inhibitors and accelerators of production of type IV and/or type VII collagen and laminin 5, which are major epidermal basement membrane constituents, notably enhance the basement membrane formation accelerating effect of matrix metalloprotease inhibitors.

It will be appreciated by those skilled in the art that while the invention has been described above in connection with particular embodiments and examples,; the invention is not necessarily so limited and that numerous other embodiments, examples, uses, modifications and departures from the embodiments, examples and use may be made without departing from the inventive scope of this application.

1. A skin basement membrane structure formation accelerating preparation comprising one or more serine protease inhibitors as active ingredients.

2. The skin basement membrane structure formation accelerating preparation according to claim 1, which further comprises one or more extracellular matrix protein production accelerators as active ingredients, and/or one or more matrix metalloprotease inhibitors as active ingredients.

3. The skin basement membrane structure formation accelerating preparation according to claim 1, wherein said serine protease inhibitor is aprotinin.

4. The skin basement membrane structure formation accelerating preparation according to claim 2, wherein said extracellular matrix proteins are one or more selected from the group consisting of Type IV collagen, Type VII collagen and laminin 5, and said extracellular matrix protein production accelerating preparation is interleukin-1, transforming growth factor-α or platelet-derived growth factor.

5. The skin basement membrane structure formation accelerating preparation according to claim 2, which is an external skin preparation.

6. A method for producing an artificial skin, comprising a step of culturing an artificial skin-forming medium, characterized by adding one or more serine protease inhibitors to said artificial skin-forming medium.

7. The method according to claim 6, characterized by further adding one or more extracellular matrix protein production accelerators and/or one or more matrix metalloprotease inhibitors to the artificial skin.

8. The method according claim 6, wherein said serine protease inhibitor is aprotinin.

9. The method according to claim 7, wherein said extracellular matrix proteins are one or more selected from the group consisting of Type IV collagen, Type VII collagen and laminin 5, and said extracellular matrix protein production accelerator is interleukin-1, transforming growth factor-α or platelet-derived growth factor.

10. An external skin preparation comprising an extract from a plant belonging to Fagaceae Fagus, a 1-acyl lysophospholipid represented by the following general formula (1) or (2), and an extract from a plant belonging to Labiatae Mentha, 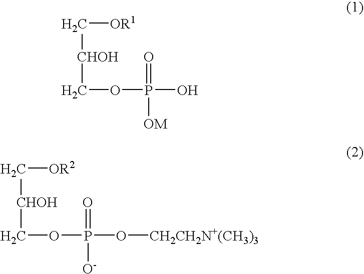
where R¹ represents a saturated fatty acid residue of 11-24 carbons or a fatty acid residue of 18, 20, 22 or 24 carbons with 1-4 unsaturated double bonds, R² represents a saturated fatty acid residue of 13-24 carbons or a fatty acid residue of 18, 20, 22 or 24 carbons with 1-4 unsaturated double bonds, and M represents H or an alkali metal atom.

11. The external skin preparation according to claim 10, which is an external skin preparation for amelioration of rough skin.

12. The external skin preparation according to claim 10, which is an external skin preparation for aging resistance.

13. The external skin preparation according to claim 10, which contains 0.001-5.0 wt % of extract from a plant belonging to Fagaceae Fagus, 0.001-1.0 wt % of a 1-acyl lysophospholipid and 0.001-5.0 wt % of extract from a plant belonging to Labiatae Mentha.

14. A skin basement membrane structure formation accelerating preparation comprising aprotinin, one or more extracellular matrix protein accelerators and one or more matrix metalloprotease inhibitors as active ingredients.

15. A method for accelerating repair and regeneration of the skin basement structure comprising administering to the skin of a patient a composition comprising one or more serine protease inhibitors as active ingredients.

16. The method according to claim 15, wherein the composition further comprises one or more extracellular matrix protein production accelerators as active ingredients, and/or one or more matrix metalloprotease inhibitors as active ingredients.

17. The method according to claim 15, wherein the serine protease inhibitor is aprotinin.

18. The method according to claim 16, wherein said extracellular matrix protein production accelerators are one or more selected from the group consisting of Type IV collagen, Type VII collagen and laminin 5, and said composition comprises interleukin-1, transforming growth factor-α or platelet-derived growth factor.

19. The method of claim 15, wherein the serine protease inhibitor is aprotinin and the composition further comprises one or more extracellular matrix protein accelerators and one-or more matrix metalloprotease inhibitors as active ingredients
